(19) United States
(12) Plant Patent Application Publication
(10) Pub. No.: US 2016/0044847 P1
(43) Pub. Date: Feb. 11, 2016

(54) Gardenia plant named 'LEETWO'

(50) ;

(71) Applicant: Robert Edward Lee, Independence, LA (US)

(72) Inventor: Robert Edward Lee, Independence, LA (US)

(21) Appl. No.: 14/121,171

(22) Filed: Aug. 11, 2014

(51) Int. Cl. A01H 5/00

(52) U.S. Cl. PLT/255

(57) ABSTRACT

A new and distinct Gardenia cultivar named ‘LEETWO’ is disclosed, characterized by distinctive small, very fragrant white, radial flowers Plant form is highly compact and dense and suitable for Southern climates. The new cultivar is a Gardenia, suitable for ornamental garden purposes.

### LATIN NAME OF THE GENUS AND SPECIES

Gardenia hybrid

### VARIETY DENOMINATION

‘LEETWO’

### BACKGROUND OF THE INVENTION

The new cultivar is a product of a planned breeding program by the inventor. This new variety, hereinafter referred to as ‘LEETWO’, was found as an openly pollinated seedling in a group of Gardenia jasminoides ‘Daisy’ (unpatented) seedling plants being grown in Independence, La. The inventor, Robert Edward Lee, discovered the seedling in Independence, La., at a research nursery during October of 2000.

After identifying the new variety as a potentially interesting selection, the inventor first organized propagation of ‘LEETWO’ by vegetative cuttings during 2004 at the same commercial nursery in Independence, La. The inventor continued confidential, controlled testing and propagation, assessing stability of the unique characteristics of this variety. Multiple generations have been reproduced and have shown that the unique features of this cultivar are stable and reproduced true to type.

### SUMMARY OF THE INVENTION

The cultivar ‘LEETWO’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘LEETWO’ These characteristics in combination distinguish ‘LEETWO’ as a new and distinct Gardenia cultivar:

1. Unique, dense plant form.

2. Tolerance for heat and disease

3. Adaptability to production and use in Southern climates.

4. Excellent fragrance.

5. Extremely compact growth

6. Deep green, shiny foliage

7. Good specimen plant

8. Good container plant

### COMPARISON TO PARENT VARIETY

‘LEETWO’ is similar in most horticultural characteristics to the seed parent variety Gardenia ‘Daisy’ unpatented. Plants of the new cultivar ‘LEETWO’ however, produce flowers that are smaller and stronger, than the seed parent variety. Additionally, plant habit is overall more compact in the new variety, than the parent, and foliage is darker green and shinier.

The pollen parent is unknown.

### COMMERCIAL COMPARISON

‘LEETWO’ can be compared to the commercial variety Gardenia hybrid ‘Leethree’, filed concurrently. Plants of the new cultivar ‘LEETWO’ are similar to plants of ‘Leethree’ in most horticultural characteristics, however, plants of the new cultivar ‘LEETWO’ produce more branches, forming a denser plant than this comparator.

### BRIEF DESCRIPTION OF THE PHOTOGRAPHS

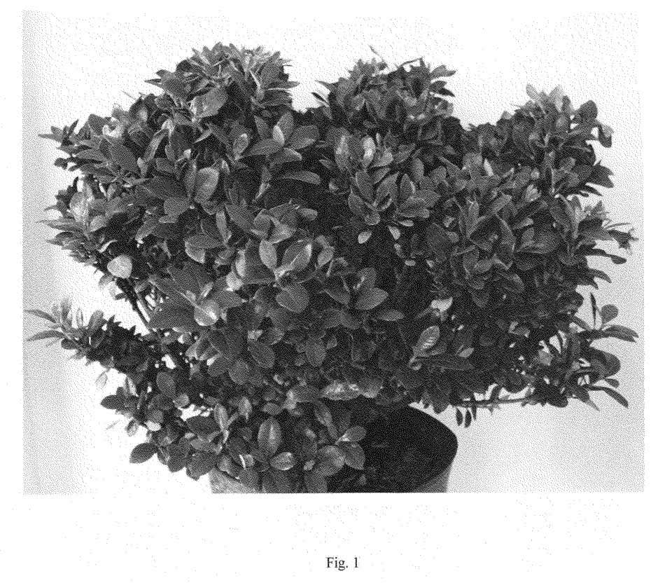

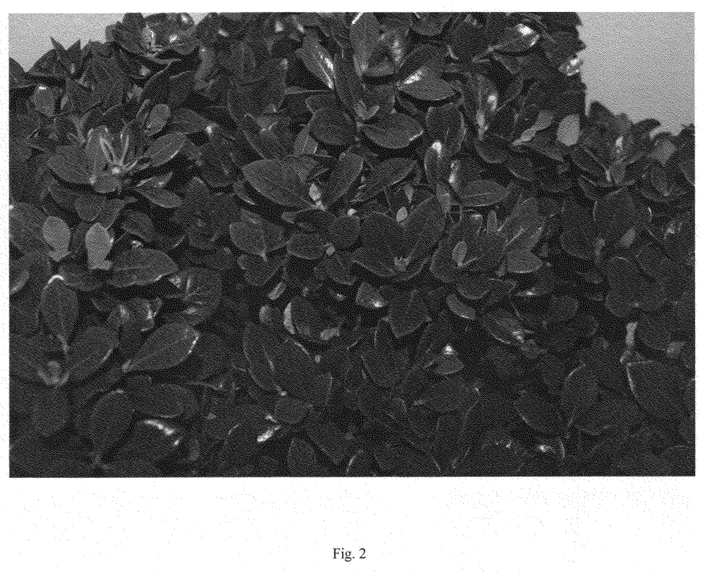

The accompanying photograph in FIG. 1 illustrates in full color a typical plant of the new variety. FIG. 2 shows a close up of foliage of ‘LEETWO’ grown outdoors in Alabama. The plant is approximately 2 years old, and is shown in a three gallon container. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘LEETWO’ plants grown outdoors in Loxley, Ala. Plants are approximately 3 years old, in a 3 gallon nursery container. Measurements and numerical values represent averages of typical plant types.
Botanical classification: Gardenia hybrid ‘LEETWO’
Propagation: Typically by semi-hardwood cuttings in Spring and Summer.
   Root description.—Woody to semi-wood. Colored near RHS Brown 200D.
Plant:
   Growth habit.—Globular shrub.
   Height.—Approximately 35 cm to top of foliar and flowering plane.
   Plant spread.—Approximately 50 cm.
   Branching characteristics.—Well branched, approximately 4 to 6 lateral branches emerge from a pinch. Branches occur at approximately 35° angles.
   Primary branches.—
   Length of primary branches.—Average 12 cm, then pinched and new branches emerge.
   Diameter of primary branches.—Approximately 0.7 cm.
   Primary branch strength.—Very strong, flexible difficult to break.
   Primary branch color.—Mature branches are colored a mottled mixture of Grey 143C, Greyed-Green 189A and Grey-Brown N199A. Young branches near Grey-Brown N199B and Brown 200B. Very small patches of Green 143A.
   Primary branch texture.—Mature branches very rough and slightly scaly. Young growth very scaly.
   Age of plant described.—Approximately 2 years.
Foliage:
   Leaf.—Type: Simple Arrangement: Opposite. At end of branches tightly spaced, to appear whorled. Average Length: Approximately 5.7 cm. Average Width: Approximately 2.6 cm. Shape of blade: Obovate. Apex: Acute. Base: Broad attenuate. Attachment: Sessile. Margin: Entire. Texture of top surface: Glabrous Texture of bottom surface: Glabrous Appearance of top surface: Very glossy Appearance of bottom surface: Matte. Color: Young foliage upper side: Near RHS Yellow-Green 144A, but darker Young foliage under side: Near RHS Yellow-Green 1448Mature foliage upper side: Near RHS Green 139A Mature foliage under side: Near RHS Green 137D.
   Venation.—Type: Pinnate. Venation color upper side: Near RHS Yellow-Green N144B Venation color under side: Near RHS Yellow-Green 144B.
Flower: Flowering measurements have not been recorded.
Reproductive organs: Measurements not recorded.
Other characteristics:
   Disease and pest resistance.—Observed to be more resistant to normal diseases and pests of Gardenia hybrid.
   Temperature tolerance.—USDA Zone 8.
   Fruit/seed production.—Not observed to date.

What is claimed is:

1. A new and distinct cultivar of Gardenia plant named ‘LEETWO’ as herein illustrated and described.
